(19) United States
(12) Patent Application Publication
(10) Pub. No.: US 2003/0078760 A1
(43) Pub. Date: Apr. 24, 2003

(54) Population pharmacokinetic modeling and analysis (PDx-POP™)

(75) Inventors: William J. Bachman, Rockville, MD (US); Sian F. Bigora, Gambrills, MD (US); Marc R. Gastonguay, Simsbury, CT (US); David Young, Ellicott City, MD (US)

Correspondence Address: ARMSTRONG, WESTERMAN & HATTORI, LLP, Suite 220, 502 Washington Avenue, Towson, MD, 21204, US

(73) Assignee: Globomax Holdings, LLC

(21) Appl. No.: 10/273,753

(22) Filed: Oct. 17, 2002

Related U.S. Application Data
(60) Provisional application No. 60/344,759, filed on Oct. 19, 2001.

(51) Int. Cl. A61B 5/00; G06G 7/48; G01N 33/48

(52) U.S. Cl. 703/11; 600/300; 702/19

(57) ABSTRACT

A biological modeling system and method for enhanced computer-aided analysis of biological response data provides information synthesized from multiple sources. An executable model of a biological system is developed from information and structures based on multiple sources. In a preferred embodiment, biological data sets are selected by a user from a first active viewer window on a user computer display. A model is created and then run using integrated pharmacokinetic software. The output is next analyzed using integrated analysis tools. Once analyzed, the model is balanced to ensure that it matches the information and structures. Once the model is created, run, and balanced, it can be used to draw attention to important relations through integrated reporting functions. This program could be run with such programs as NONMEM®.

### CROSS REFERENCE TO RELATED APPLICATIONS

This application claims priority to U.S. provisional patent application serial No. 60/344,759 filed Oct. 19, 2001.

### BACKGROUND OF THE INVENTION

1. Field of the Invention

This invention pertains generally to data processing from biological systems. More specifically, the present invention is a pharmacokinetics system comprising seamlessly integrated computer modeling and display tools which provides a vastly expedited, dynamic and interactive modeling and analysis system.

2. Description of the Related Art

Many prior art methods of obtaining biological process data require time consuming laboratory experiments. Data is usually obtained from live animal experiments and clinical trials which are costly and provide many difficult-to-control variables that may mask biochemical activities which are the response of interest. The complexity of the information does not always provide a clear and consistent picture from which accurate conclusions can be drawn.

In an effort to provide more clear and consistent test results, clinical trials are typically designed to isolate a single variable, and use a placebo control group as a baseline from which the variable is measured. Observations from a clinical trial are used to attempt to draw conclusions from apparent differences between the control group and the experimental group. These observations, however, rarely take into account the multi-variable dynamic nature of the patients, either individually or as a group. Such variations are, however, reflected in the data and require large test populations to deal with in an appropriate statistical manner.

Unfortunately, and owing to the single-variable nature of the drug development business, which was designed to clarify the results of the experiments, the reported data results in a great degree of uncertainty. Each study provides a very limited view of the complete living system. Various diverse factors, including diet, age, sex, unexpected interactions with other drugs or compounds, and many other factors may alter the results of the experiment. Ultimately, the different studies fail to provide a complete picture of the entire biological system, by design.

A typical cycle for a clinical trial requires years of work. Designing the trial may take six months, performance of the trial may take a year, and analysis of the results may take yet another six months. After years of testing, the results may still be suspect. Additionally, a trial may be one of several ongoing trials necessary to address the variables associated with a particular area of investigation.

Only after numerous costly trial-and-error clinical trials, and constant redesigning of the clinical use of the drug to account for lessons learned from the most recent clinical trial, is a drug finally realized that has adequate safety and efficacy. This process of clinical trial design and redesign, multiple clinical trials and, in some situations, multiple drug redesigns, requires much time and money. Even then, the effort may not produce a marketable drug.

Owing to the cost and difficulty of the experiments, drugs that may be cost-effectively researched and developed using this type of modeling are few. They generally include either refinements to existing drugs, or an attempt to develop a drug for a new application that was inferred from observations made during previous clinical trials and experiments. The enormous risk prevents the development of pharmaceuticals for anything but an extremely large segment of the population. Biological abnormalities which may be treatable by a drug may not be explored, because the potential market for the drug does not justify the expenditure of resources necessary to design, test, and obtain approval for the drug. Even with large market segments, development is extremely speculative. In summary, the cost of drug development is very high and difficult to justify except for the largest of patient populations and lowest of risks.

A number of in vitro or cell culture-based methods have been described for identifying compounds with a particular biological effect. From these trials and experiments, data is obtained which usually focuses on a more specific part of the biological system, and avoids some of the variables that cannot otherwise be controlled. While conclusions may be drawn by assimilating experimental data and published information, it is difficult, if not impossible, to synthesize the relationships among all the available data and knowledge. Incorporation of the many diverse variables of living systems can be misleading.

Studies of the pharmacokinetics or pharmacodynamics of drugs help us learn about the variability in drug disposition and effects in a population. For the purpose of this disclosure, pharmacokinetics will be understood to be the study of the time course of a drug and its breakdown products after administration to the body in terms of absorption, resorption, distribution, metabolism and elimination. Pharmacodynamics will be understood to be the study of the relationship of drug concentration to pharmacologic effects. The word “drug” will also be understood to comprise a chemical or chemical composition having one or more molecular constituents which interact with living organisms. Pharmacokinetic and pharmacodynamic information can be used to treat patients at safe and effective doses if these studies enable a person to infer pharmacologically important characteristics of the treatment population. Useful studies may, for exemplary purposes, but not limited thereto, predict the structure, form, dose and other aspects of the administration of a drug in a patient to maximize the drug's efficacy while minimizing the risk of toxicity.

Pharmacokinetic studies are used to assess the systemic exposure of administered drugs and factors likely to affect this exposure. The studies are desirably carried out in a well-controlled clinical environment. Samples are collected on each of the study subjects, and concentration-time data are analyzed to derive parameters such as the observed maximum concentration, Cmax, and the area under the concentration-time curve, AUC.

Population pharmacokinetics has been defined as “the study of the sources and correlates of variability in drug concentrations among individuals who are the target patient population receiving clinically relevant doses of a drug of interest”. Methods of population pharmacokinetics are indicated when kinetic data of different individuals have to be combined and when the average behavior of a population is used to predict an individual kinetic.

Diversity of patient characteristics in population pharmacokinetic studies and large sample sizes allow for the exploration of possible problems and factors that may affect drug exposure and for testing of interactions among factors. The better the pharmacokinetic variability can be explained with these factors, the better drug exposure can be predicted for patient sub-populations or even individuals, and the more appropriately a dose can be tailored. Population pharmacokinetic studies can better address simultaneously many issues that normally are the objectives of separate traditional pharmacokinetic studies, thereby reducing the need for some of these studies. Significant savings in time and resources can be gained not only in terms of the sizes and time lines of the early development programs, but also in terms of rational and efficient large scale efficacy and safety clinical studies. Population pharmacokinetic studies do not require frequent blood sampling from individual patients, and concentration-time data can be directly modeled.

From a regulatory standpoint, a well designed and conducted population pharmacokinetic study can enhance a submission by better depicting the overall drug-concentration relationship. In fact, more and more reviewers are recommending the inclusion of a population pharmacokinetic analysis. Findings from well-conducted population pharmacokinetic studies can be used to support critical labeling claims. In addition, the FDA in “Guidance for Industry: Providing Clinical Evidence for Human Drug and Biological Products” issued in May 1998, provides sponsors of human drugs an opportunity to utilize population pharmacokinetic results as critical evidence in support of a single adequate and well-controlled clinical investigation in seeking regulatory approval of a new use of a drug. In view of the enormous benefits obtained, pharmacokinetic and pharmacodynamic information are the basis of modem pharmacotherapy.

Population pharmacokinetics has also come to mean the application of nonlinear mixed-effects modeling to any pharmacokinetic data sampled sparsely such that AUC and Cmax cannot be computed for each individual (even non-human) using that individual's data alone. The FDA Guidance offers another definition of population pharmacokinetics that explicitly contains the association with hierarchical modeling: “These models, their parameter values, and the use of study designs and data analysis methods designed to elucidate population pharmacokinetic models and their parameter values, are what is meant by population pharmacokinetics.”

The statistical analysis of pharmacokinetic data addresses time-dependent repeated measurements of drug of concentrations in various organs of the body, with the goal to describe the time course and to determine clinically relevant parameters by modeling the organism through compartments and flow rates. The mathematical solution is a system of differential equations with an explicit solution for most of the one or two compartment models. Otherwise, numerical solutions have to be used. Intrinsic pharmacokinetic parameters include area under the curve (AUC), clearance, distribution volume, halftime, elimination rates, minimum inhibitory concentrations, etc. Numerous computer programs for linear and simple non-linear regression methods have been reported. Fitting procedures have been programmed on spreadsheet platforms. Software packages have been released that require special training. Other package-specific software has been suggested, using standard statistical systems. As may already be recognized, the data source files and output are handled quite differently among the various components, which makes the use of each of the tools exceedingly cumbersome and difficult. This is in spite of the fact that the computation and determination of statistical variability of parameters are critical for the continued development, use, and assessment of computational systems in pharmacokinetics.

NONMEM® software (the Regents of the University of California, Oakland, Calif.) is one most preferred, commercially available and widely known package concerned with the development of data analysis techniques and exportable software for fitting nonlinear mixed effects (statistical regression-type) models. These techniques are particularly useful when the data are population pharmacokinetic/pharmacodynamic data, and when there are only a few PK/PD measurements from some individuals sampled from the population, or when the regression design varies considerably between individuals. However, increasingly, the techniques are also being used advantageously with better-designed experimental type data. As the software evolves, it reflects tested methodological and programming improvements.

Xpose is a most preferred, commercially available S-PLUS® (Insightful Corporation, Seattle, Wash.) based model building aid for population analysis using NONMEM. It facilitates data set checkout, exploration and visualization, model diagnostics, candidate covariate identification and model comparison. Data set checkout includes visualization of the observed variable(s), covariates and plots to reveal errors in the data file. Model diagnostic plots includes the usual residual plots but also includes plots to check the validity of assumptions specific to non-linear mixed effects models. Data exploration is also done by various plots but also includes auxiliary screening analyses such as stepwise generalized additive modeling (GAM) and tree based modeling. The stability of the GAM results with respect to covariate model selection as well as the impact of influential individuals and certain types of covariate interactions can be explored using a bootstrap re-sampling procedure.

Other software packages and programs are also available which further complement the generation of pharmacokinetic and pharmacodynamic models and analyses, as already noted herein above, and include such programs as Microsoft Excel which provides graphing and other analysis of data. Unfortunately, each is an island unto itself, with little integration or file import and export capability provided. Further, the ability to selectively extract and analyze limited data sets is lacking. What is needed then, is an alternative system and method which efficiently integrates tools that reveal and convey information regarding complex biological systems.

### SUMMARY OF THE INVENTION

The present invention provides a method and apparatus which allows critical integrated evaluation of data and hypotheses. The model can be built to simulate individual patients or specific groupings of patients, or the general population as a whole. By providing individual patient simulations, individual susceptibility and environmental factors can be directly linked to the biology and clinical outcomes. Specific grouping patient simulations also provides a way of exploring patterns of patient-level factors that may influence biologic behavior.

In a first manifestation, the invention is, in combination, a computer executable pharmacokinetic model of a biological system, a computer executable data editor, a computer executable report generator, a data stream representing a chemical level within the biological system at a particular time period, a computer system including a memory and a processor, and a computer executable integrator. The integrator has: a means to locate at least one component of the computer executable pharmacokinetic model of a biological system; a means to establish a protocol for a control stream which will execute the computer executable pharmacokinetic model of a biological system; a means for generating the control stream responsive to the establishing means; a means to distinguish the computer executable data editor; a means to control execution of the computer executable data editor; a means to identify the computer executable report generator; and a means to manage execution of the computer executable report generator.

In a second manifestation, the invention is, in combination, a computer executable pharmacokinetic model of a biological system, a computer executable data editor, a computer executable report generator, a data stream representing a chemical level within the biological system at a particular time period, a computer system including a memory and a processor, and a computer executable integrator having: a means to locate at least one component of the computer executable pharmacokinetic model of a biological system, the locating means compatible with NONMEM; a means to establish a protocol for a control stream which will execute the computer executable pharmacokinetic model of a biological system, the establishing means compatible with NONMEM and responsive to the locating means; a means for generating the control stream responsive to the establishing means; a means to distinguish the computer executable data editor; a means to control execution of the computer executable data editor responsive to the distinguishing means; a means to establish a conversion protocol operative to translate the data stream between a data format used by the computer executable pharmacokinetic model of a biological system and a data format used by the computer executable data editor, the conversion protocol responsive to the locating means; a means to identify the computer executable report generator that is compatible with S-Plus; a means to manage execution of the computer executable report generator that is compatible with S-Plus and responsive to the identifying means; and a means to establish a conversion protocol operative to translate the data stream between a data format used by the computer executable pharmacokinetic model of a biological system and a data format used by the computer executable report generator, the conversion protocol responsive to the locating means.

In a third manifestation, the invention is, in combination, a computer executable pharmacokinetic model of a biological system, a computer executable data editor, a computer executable report generator, a data stream representing a chemical level within said biological system at a particular time period, a computer system including a memory and a processor, and a computer executable control center having: a means to launch each of the computer executable pharmacokinetic model of a biological system, the computer executable data editor, and the computer executable report generator; and a means for displaying real-time progress of the computer executable pharmacokinetic model of a biological system.

In a fourth manifestation, the invention is a method of processing pharmacokinetic data having enhanced data management and exploration. The steps include: producing a first data set representing a time course of a chemical within at least one living organism; selecting at least one criterion for splitting the first data set; splitting the first data set into a first data subset and a second data subset in accord with the selected at least one criterion; developing a model of the time course of the chemical within at least one living organism; generating a second data set representing the time course of the chemical within at least one living organism in accord with the model; comparing second data set with first data subset; analyzing the developed model responsive to the comparing step; revising the model responsive to the analyzing step; generating a third data set representing the time course of the chemical within at least one living organism in accord with the revised model; contrasting the third data set with second data subset; and validating the revised model responsive to the contrasting step.

### OBJECTS OF THE INVENTION

A first object of the invention is to provide a system and method for modeling biological systems. A second object of the invention is to provide a system and method for modeling biological systems in a manner reflecting the dynamic and multi-variable nature of the systems. A third object of the invention is to provide a method for drug development which provides an enhanced user interface and new back-end reporting tools to improve the users' experience. Another object of the invention is to include additional analysis tools in seamless integration with the preferred core product. Yet another object of the invention is to facilitate problem specification and model definition. An additional object of the invention is to upgrade the visualization and reporting of NONMEM results. These and other objects are achieved in the present invention, which may be best understood by the following detailed description and drawings of the preferred embodiment.

### BRIEF DESCRIPTION OF THE DRAWINGS

FIG. 1 illustrates a preferred method which demonstrates the teachings of the present invention.

FIG. 2 illustrates a preferred apparatus for implementing the preferred method of FIG. 1.

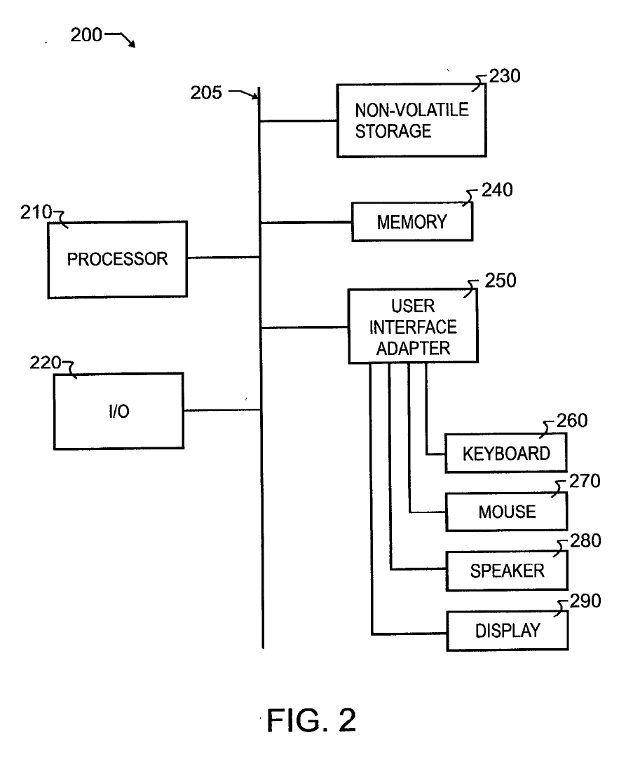

FIG. 3 illustrates the sequences associated with a preferred Model/Run Tab

FIG. 4 illustrates the sequences associated with a preferred Output Tab

FIG. 5 illustrates the sequences associated with a preferred Menu Bar

### DESCRIPTION OF THE PREFERRED EMBODIMENT

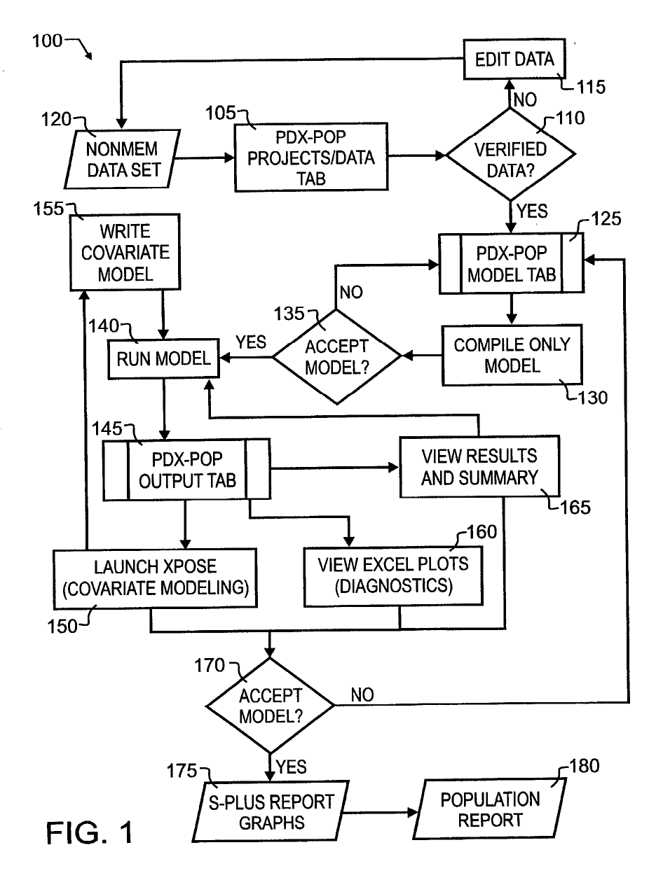

A preferred embodiment integrated population pharmacokinetic modeling and analysis method 100 is illustrated in FIG. 1 by simplified block diagram. As illustrated therein, preferred method 100 seamlessly integrates existing software packages and tools to expedite the iterative process of population pharmacokinetic modeling and analysis. Working in concert with preferred NONMEM, S-PLUS Analytic Software and MS Excel, preferred method 100 delivers optimal flexibility, increased efficiency and added functionality.

Preferably, the user interface will comprise software for driving a menu-driven, multi-window graphical interface which will allow the user to easily manipulate and analyze data in one or more simultaneous viewer windows. In a preferred embodiment, the user interface is adapted to provide the look and feel of an Internet browser interface, a Windows 95/98/2000/ME/NTXP interface, a KDE interface, or other X-Windows type interface.

Preferred method 100 follows a three-tab paradigm that represents the major project functions, and which proceeds in the same order in which a user will preferably develop a model based on either imported or entered data. The tabs, or suitable alternatives and equivalents as will be recognized by those skilled in the art, will be displayed upon a computer monitor or other display 290. A user or operator can move between the three tabs by clicking on the respective “Project/Data”, “Model/Run” and “Output” tabs that may, for exemplary purposes only, be found in a control center located in the top half of the display 290. The bottom portion of display 290 may contain an “Output Window” in which the data-splitting output and NM-TRAN and NONMEM runtime outputs may be displayed.

The first step, preferably represented as a “file tab” or other equivalent on computer display interface 290 and described herein as step 105, is the PDx-POP projects/Data step. The purpose of the project function of step 105 is to assign a project name and/or number, create or select a project directory and switch between different projects.

From the data function of this step 105, a user may select or examine data of interest, and optionally split a data set into a data subset comprising test individuals or test samples that are selected by the user at will or randomly. Most preferably, the operator may also optionally compile single or multiple data subsets, and select the data records at will from other existing data sets or subsets. A particular benefit of this capability is the ability for a user to readily create an index and validation data set for modeling and model validation steps. Additional data related functions are preferably initiated from this step 105, and may include such actions as data verification step 110, data editing step 115, which may optionally include launching a separate software package such as Microsoft Excel or the like, importing and selecting of external data sets such as NONMEM data set 120, and viewing or plotting the data file, preferably by launching Excel or the like to facilitate viewing and plotting the data in useful formats. In the data editing step 115, the data will most preferably be exportable or edited directly as a NONMEM data set. Consequently, there is no extra data translation or conversion required between the editing and operating upon or from data set 120.

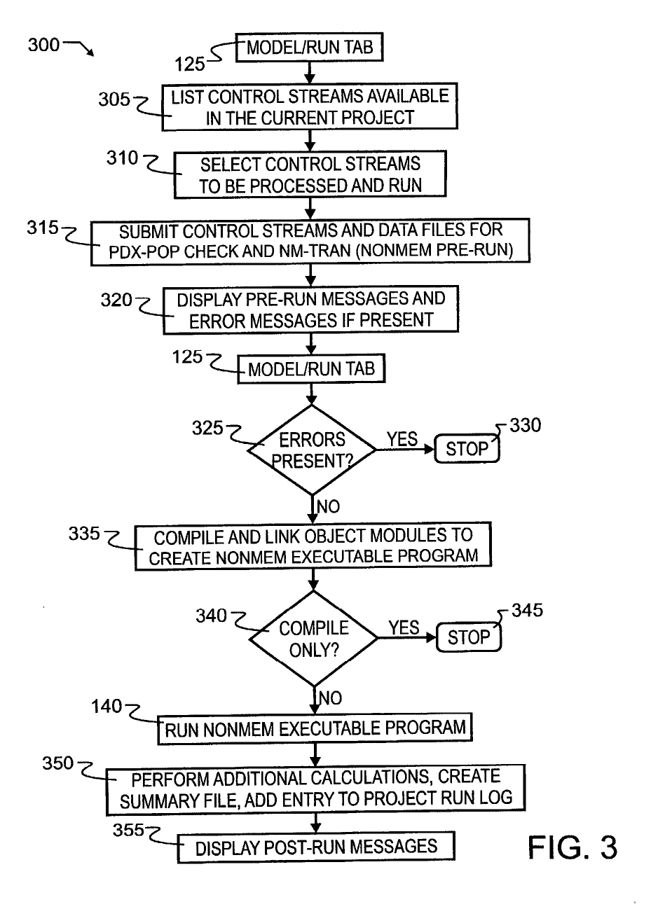

The second major processing function, preferably represented as a second “file tab” or other equivalent on computer display interface 290 as model tab 125, is to perform modeling tasks. The modeler performs control stream related functions such as editing or creating new control streams, selecting control streams to be run, and then debugging and re-running the control streams. Control streams are files containing a list of specifications and instructions for an individual model “run” used in modeling population data. Single runs, or multiple sequential runs commonly referred to as batches, may therefore be performed in the preferred method 100. From model tab 125, a model may be compiled at step 130, accepted at step 135 and run at step 140, or rejected at step 135, thereby returning control back to model tab 125. Through the modeling interface accessed by clicking on model tab 125, which will be explained in further detail with regard to the model/run process illustrated in FIG. 3, an operator will preferably have the ability to create, sort, view, edit, copy or delete control stream files; expedite the creation of control stream files with a standard template; point-and-click to easily select, prioritize and compile the control streams to be run, either individually or in batches; perform error checking of control streams prior to batch runs; and view progress before analysis is complete through a “View Intermediate Results” feature.

The third major step, also preferably represented as a “file tab” or other equivalent on computer display interface 290, is to assess and report the results through the PDx-POP output tab 145. Output tab 145 provides access to a log of all the runs that have been made in the current project. This tab also provides the user with access to a variety of different output options for all the runs made in the project, including: viewing the full NONMEM output, viewing an abbreviated summary of the model results, and viewing and plotting the data in NONMEM-generated tables in step 165; performing modeling and diagnostic tasks using Xpose and S-PLUS or the like in step 150; viewing diagnostics using Excel or similar plots in step 160; and creating report-ready graphs with S-PLUS or Excel at steps 175 and 180. While the S-Plus report graphs 175 and population report 180 are shown after the step of accepting the model 170, which would be the typical sequence, it will be understood that these reports may be produced prior to or subsequent to the acceptance of the model at step 170 without altering the intent of the invention. The Xpose package may be used for covariate modeling, which most preferably will permit a user to write a covariate model in step 155 for use directly in subsequently running the model at step 140.

Steps 135 and 170, which are decision points regarding model acceptance, are important to the balancing of the model. Once the model is created in the modeling tool, the model must be run and modified to accurately reflect observed phenomenon. Modifying in the prior art is extremely time consuming and labor intensive, requiring tedious input to represent knowledge not available in the stored models. This knowledge alters the model from one with less real world correlation to one that accurately reflects the clinical behaviors. The modifying process can help to identify inconsistencies in knowledge stored in the database.

Before and after the model is run, each observable characteristic or data item should be checked against corresponding real world data. For example, a particular piece of literature may deal with a particular biological system which is self contained within a particular level of the model. This level entity may be checked for accuracy against the real world information disclosed in the literature. Changes may be made to the model repeatedly.

Once the model exhibits reasonable performance, the values of the outputs are re-interpreted and mapped into values that correlate with actual clinical outcomes. The model is then systematically run and tested using a set of matrices on which clinical and experimental data are recorded. The model is run repeatedly, systematically altering the various input data and recording the various internal outputs of the model, to ensure that the outcomes of the model make sense. A redesign and/or a re-modifying of certain portions of the model may need to be made at this point to ensure proper behavior under the various key situations of interest.

The present invention offers much advantage over the prior art, since the various individual and isolated components and tools used for processing pharmacokinetic data in the prior art are integrated, and available in real time on user-selected data sets or subsets. This enables much more rapid modeling and testing than was heretofore possible.

The present method in the preferred embodiment is stored as a computer instruction set or software program in non-volatile storage 230 or through a network or remote location via input/output (I/O) 220. The instructions are most preferably executed by a processor 210 utilizing memory 240 for data storage required during program execution. Processor 210 may take many forms, including a single microprocessor or dedicated controller, a central processing unit (CPU), one or more sets of parallel processors, one or more reduced instruction set controllers (RISC), distributed processors which are either local or distributed through a network, a neural network, or any of the others of the myriad of known processing techniques. The results are preferred to be conveyed to a user using standard user interface technology through one or more user interface adapters 250 which in turn provide the electronic communication a keyboard 260, mouse 270, speaker 280 and display 290 and processor 210. Nevertheless, the use of a particular hardware, apparatus or structure is not critical to the invention, provided there is an efficient means of carrying out the requisite steps of the invention.

Advantageously, according to a preferred embodiment, preferred integrated population pharmacokinetic modeling and analysis method 100 is written using a programming language that allows for platform independence such that it may be executed on any of a variety of user computing apparatus 200 having different operating systems. As known in the art, Fortran, Java and Perl are examples of programming languages optimized for cross-platform computing, though other languages will be recognized as suitable for the execution of the preferred method 100.

A preferred model and run method 300 is illustrated in more detail in FIG. 3. To begin the development of a model, a user will click the “Model/Run” tab 125. All NM-TRAN control streams that are present in the current project directory and that comply with the control stream naming conventions and modifications for the preferred method 100 will be listed in a window entitled “Control Streams Available” at step 305. The user will next select control streams to be processed and run in step 310, and may, during this step, create, edit, copy and delete control streams. Next, the user will submit control streams and data files for check by preferred method 100 and also by NM-TRAN, through a NONMEM pre-run at step 315. Preferred method 100 will provide for a display of pre-run messages and error messages, if any are present, at step 320. The operator has the option at step 315 of submitting the control streams for a “Compile Only” execution that allows pre-checking of runs for errors, as shown at step 130 in FIG. 1. Two additional options will preferably be provided. A “View Intermediate Results” option allows easy viewing of intermediate NONMEM output, preferably on a separate window or pane, and the “Abort Run” option allows early termination of a model run that is determined to be unnecessary or flawed. After the pre-run and error messages are displayed at step 320, the user will be returned to model/run tab 125 and will make a decision at step 325 regarding whether errors are present. If so, the user will have the opportunity to stop at step 330, or to return to an earlier step such as step 305 to fix the error. If the control streams and data files are ready for running, the compiling and linking of object modules to create NONMEM executable program is carried out at step 335. If the operator selected the “Compile Only” option described herein above, processing will stop at step 345, in effect returning control back to preferred method 100. Otherwise, model and run method 300 will run the NONMEM or similar executable program used to perform modeling at step 140. When this program has been executed, method 300 will perform additional calculations, create a summary file, and add an entry to the project run log at step 350. Finally, the method will display post-run messages at step 355.

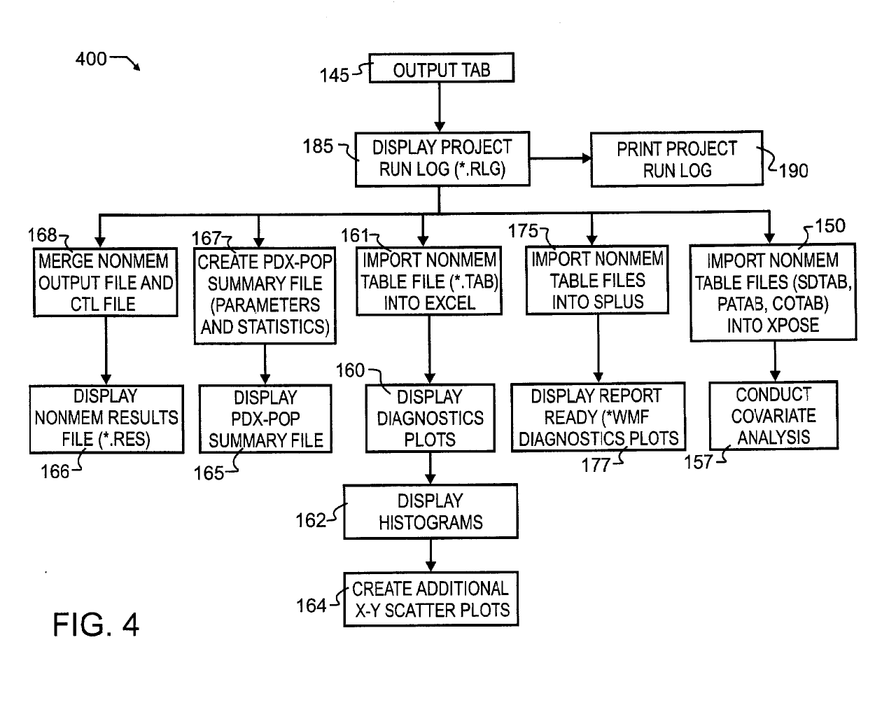

A preferred output interface and method 400 is illustrated in FIG. 4. This method, like method 300, is merely an exemplary and illustrative preferred method, which will enable those skilled in the art to infer many alternatives too numerous in design and implementation to individually recite herein. Nevertheless, these alternatives will be clearly understood to be contemplated herein. As illustrated, once a model has been executed or run at step 140, the user will preferably be taken to or will access output tab 145. To view the results of a run, the operator will click on the “Output Tab” 145 of a user interface control center. Output tab 145 displays a summary of all the runs made in the current project in step 185. These may be stored having a common suffix, such as “*.rlg”. To view the model results, the operator will, in the preferred embodiment, first highlight the desired run with mouse 270, and then select from five output options described herein below by checking the desired box and clicking a “View Output” button. The functions of the “Output” Tab are to display the current active project name and number to identify the output available, as shown in step 185. Multiple projects can concurrently be active. Most preferably, this step will also display a chronological project run log with summarized details of all models that have been run from project inception to last model run. The details might, for exemplary purposes, include the run number, data file used, run status indicators and a model description that allow a quick assessment of the model run, and comparison between model runs. The operator may elect to print the project run log, as shown in step 190, and/or incorporated in the final project report of steps 175 and 180. Additionally, more complex forms of output can be automatically generated by the preferred method 100, NONMEM, S-Plus, Microsoft Excel and other tools and packages, and may be displayed. As shown at steps 168 and 166, the operator may display complete NONMEM output with appended control stream, run start/stop times and compiler information. In steps 167 and 165, the operator may display abbreviated summary files with model parameter estimates extracted from NONMEM output and additional statistics including % CV's, standard errors, relative standard errors, and 95% confidence intervals. The operator may launch Microsoft Excel and automatically import a NONMEM output table having the suffix “*.tab” in step 161, and generate diagnostic plots in step 160. Histograms may be displayed in step 162, and the operator may create additional x-y scatter plots in step 164. As an alternative to Excel of steps 160-164, the operator may elect to launch S-Plus and automatically import NONMEM output table files in step 175, and generate report ready “*.wmf” diagnostic plots in step 177. As yet another alternative, the operator may launch Xpose, automatically import NONMEM output tables such as sdtab, patab, and cotab in step 150, and either generate diagnostic plots or use the imported tables to perform a variety of population modeling tasks available from a large menu of items including conducting covariate analysis as shown in step 157. The preferred output interface and method 400 has much advantage over the prior art by enabling an operator to post-process NONMEM output, in fewer steps, with minimal input. The method more efficiently utilizes outputs when the operator is performing analyses, applying all S-PLUS and MS Excel functions. The output is produced in formats designed to streamline the analysis and reporting process, and to provide compatibility with other systems. In the preferred embodiment, method 400 will use standard NONMEM results with additional information on the control stream. Method 400 will also preferably provide enhanced output summary results, including final estimates for theta, omega and sigma and their associated standard deviation, % CV, % RSE and confidence intervals. Method 400 will most preferably implement fully functional S-PLUS or MS Excel tables, and may generate automatic S-PLUS or MS Excel standard plots.

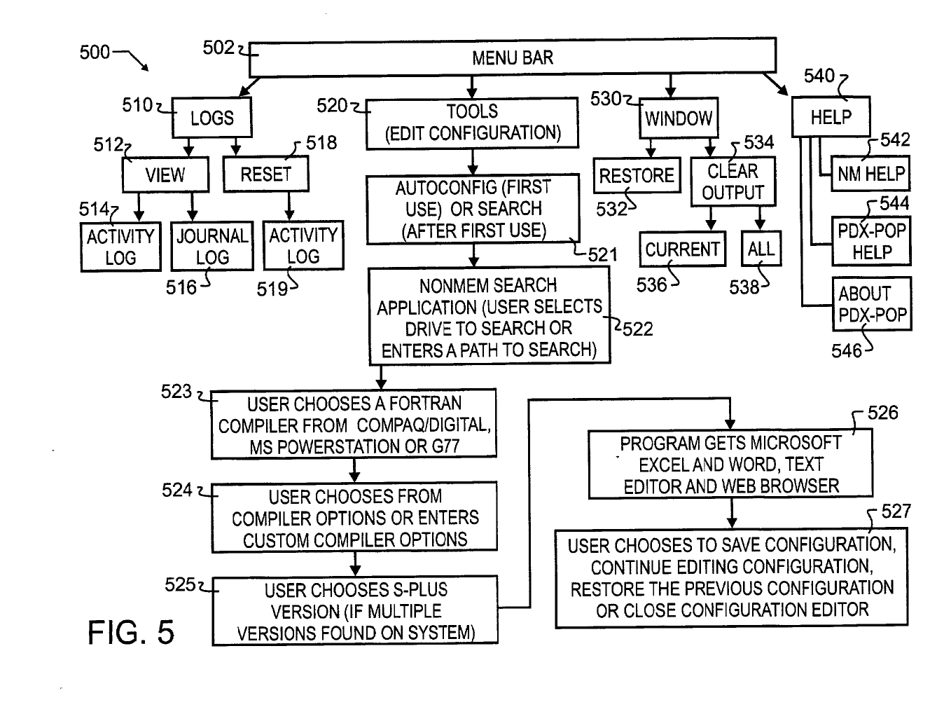

In addition to the three “Tab” windows of the preferred method 100 control panel, there are menu bar functions that are preferably made available from menu bar 502, located generally at the top of the application window. These functions will preferably include “Logs” 510, “Tools” 520, “Window” 530, and “Help” 540, each detailed in FIG. 5 as a part of method 500. The functions of the “Logs” menu 510 includes viewing in step 512, including activity log 514 or journal log 516. Activity log 514 is a chronological listing of all activities that have generated output in the current project. Journal log 516 contains the same entries as the project run log and also accepts additional user-entered entries that can be used to help document the analysis or contain additional user notes. A second function of the “Logs” menu 510 is to reset, in this embodiment specifically emptying the activity log at step 519. The “Logs” menu 510, together with the internal and automatic logging, provides tracking, automatically generating an audit trail. As will be understood, this is obtained by enabling an operator to view, print or save as file the run log, thereby tracking all steps in the analysis. The preferred method 500 will maintain an electronic journal associated with the analysis to record important information, such as comments on runs, models used, decisions made on outputs, etc.

The function of “Tools” menu 520 is to allow configuration or re-configuration of the preferred method 100 with respect to the definition of the third-party software that interacts therewith, including for example a text editor, Microsoft Excel and Word, the NONMEM directory, a Fortran compiler and options, S-Plus and associated directory, the Xpose file location and a web browser. An auto-configuration process 521 is used that chooses configuration options from the Windows Registry when possible, and user-input when not appropriate. In accord with method 500, at step 522 a search for the NONMEM application is initiated. The user may select the drive to search or will instead enter a path to search. At step 523 the user will select a Fortran compiler. As illustrated, the options may include compilers from Compaq/Digital, MS Powerstation or G77, though these are only listed as exemplary. At step 524 the user will be asked to select from compiler options or enter custom compiler options. At step 525, the user will choose an S-Plus version, if multiple versions are found on the system by the auto-configuration technique. At step 526, the method will locate Microsoft Excel, Word and/or another preferred text editor, and a web browser. Finally, at step 527, the user selects between saving the configuration, continuing editing the configuration, restoring the previous configuration, or closing the configuration editor. Most preferably, within the configuration processes of method 500 are appropriate data conversions and protocols for communication of data between each of the integrated components. The selection of the various protocols and data conversions that will be required will, of course, be dependent upon the particular software packages supported, as will be apparent to those skilled in the art after a review of the present disclosure.

The “Window” menu 530 functions are to restore the control console and output windows to default locations in step 532, or to clear the output window(s) from step 534 for either the current active output window in step 536 or all output windows in step 538. In preferred method 100, if runs have been made in more than one project during the current session, multiple output windows may be displayed and selected from project name labeled tabs.

The “Help” menu 540 has the following options. NM help step 542 displays an HTML version of the NONMEM help files in a web browser window. PDx-Pop help step 544 displays an HTML version of the manual for the preferred method 100 in a web browser window. About PDx-Pop step 546 displays PDx-Pop version information.

Having thus disclosed the preferred embodiment and some alternatives to the preferred embodiment, additional possibilities and applications will become apparent to those skilled in the art without undue effort or experimentation. Therefore, while the foregoing details what is felt to be the preferred embodiment of the invention, no material limitations to the scope of the claimed invention are intended. Further, features and design alternatives that would be obvious to one of ordinary skill in the art are considered to be incorporated herein. Consequently, rather than being limited strictly to the features recited with regard to the preferred embodiment, the scope of the invention is set forth and particularly described in the claims hereinbelow.

We claim:

1. In combination, a computer executable pharmacokinetic model of a biological system, a computer executable data editor, a computer executable report generator, a data stream representing a chemical level within said biological system at a particular time period, and a computer system including a memory and a processor, wherein the improvement comprises a computer executable integrator having:
a means to locate at least one component of said computer executable pharmacokinetic model of a biological system;
a means to establish a protocol for a control stream which will execute said computer executable pharmacokinetic model of a biological system;
a means for generating said control stream responsive to said establishing means;
a means to distinguish said computer executable data editor;
a means to control execution of said computer executable data editor;
a means to identify said computer executable report generator; and
a means to manage execution of said computer executable report generator.

2. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said computer executable integrator further comprises a means to establish a conversion protocol operative to translate said data stream between a data format used by said computer executable pharmacokinetic model of a biological system and a data format used by said computer executable data editor, said conversion protocol responsive to said locating means.

3. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said computer executable integrator further comprises a means to establish a conversion protocol operative to translate said data stream between a data format used by said computer executable pharmacokinetic model of a biological system and a data format used by said computer executable report generator, said conversion protocol responsive to said locating means.

4. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said means to establish a protocol for a control stream is responsive to said locating means.

5. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said means to control execution of said computer executable data editor is responsive to said distinguishing means.

6. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said means to control execution of said computer executable report generator is responsive to said identifying means.

7. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said means for generating said control stream responsive to said establishing means generates a plurality of control streams for executing a plurality of data streams representing chemical levels within said biological system.

8. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, further comprising a means to choose a computer programming language compiler.

9. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said computer executable pharmacokinetic model of a biological system further comprises NONMEM.

10. The combination of a computer executable pharmacokinetic model of a biological system, a computer executable data editor, a computer executable report generator, a data stream representing a chemical level within said biological system at a particular time period, a computer system including a memory and a processor, and a computer executable integrator of claim 1, wherein said computer executable report generator further comprises S-Plus.

11. In combination, a computer executable pharmacokinetic model of a biological system, a computer executable data editor, a computer executable report generator, a data stream representing a chemical level within said biological system at a particular time period, and a computer system including a memory and a processor, wherein the improvement comprises a computer executable integrator having:
a means to locate at least one component of said computer executable pharmacokinetic model of a biological system, said locating means compatible with NONMEM;
a means to establish a protocol for a control stream which will execute said computer executable pharmacokinetic model of a biological system, said establishing means compatible with NONMEM and responsive to said locating means;
a means for generating said control stream responsive to said establishing means;
a means to distinguish said computer executable data editor;
a means to control execution of said computer executable data editor responsive to said distinguishing means;
a means to establish a conversion protocol operative to translate said data stream between a data format used by said computer executable pharmacokinetic model of a biological system and a data format used by said computer executable data editor, said conversion protocol responsive to said locating means;
a means to identify said computer executable report generator compatible with S-Plus;
a means to manage execution of said computer executable report generator compatible with S-Plus responsive to said identifying means; and
a means to establish a conversion protocol operative to translate said data stream between a data format used by said computer executable pharmacokinetic model of a biological system and a data format used by said computer executable report generator, said conversion protocol responsive to said locating means.

12. In combination, a computer executable pharmacokinetic model of a biological system, a computer executable data editor, a computer executable report generator, a data stream representing a chemical level within said biological system at a particular time period, and a computer system including a memory and a processor, wherein the improvement comprises a computer executable control center having:
a means to launch each of said computer executable pharmacokinetic model of a biological system, said computer executable data editor, and said computer executable report generator; and
a means for displaying real-time progress of said computer executable pharmacokinetic model of a biological system.

13. The combination of a computer executable pharmacokinetic model of a biological system, a computer executable data editor, a computer executable report generator, a data stream representing a chemical level within said biological system at a particular time period, and a computer system including a memory and a processor of claim 12, further comprising a means to choose between a compile-only operation of said computer executable pharmacokinetic model of a biological system and a full modeling operation of said computer executable pharmacokinetic model of a biological system.

14. A method of processing pharmacokinetic data having enhanced data management and exploration, comprising the steps of:
producing a first data set representing a time course of a chemical within at least one living organism;
selecting at least one criterion for splitting said first data set;
splitting said first data set into a first data subset and a second data subset in accord with said selected at least one criterion;
developing a model of said time course of said chemical within said at least one living organism;
generating a second data set representing said time course of said chemical within said at least one living organism in accord with said model;
comparing said second data set with said first data subset;
analyzing said developed model responsive to said comparing step;
revising said model responsive to said analyzing step;
generating a third data set representing said time course of said chemical within said at least one living organism in accord with said revised model;
contrasting said third data set with said second data subset; and
validating said revised model responsive to said contrasting step.

15. The method of processing pharmacokinetic data having enhanced data management and exploration of claim 14, further comprising the steps of:
examining individual datum within said first data set visually; and
editing said individual datum.

16. The method of processing pharmacokinetic data having enhanced data management and exploration of claim 15, wherein said examining and editing step further comprising the steps of:
launching a spreadsheet program; and
activating data editing features provided within said spreadsheet program.

17. The method of processing pharmacokinetic data having enhanced data management and exploration of claim 14, further comprising the steps of:
creating a report of said pharmacokinetic data processing including data representations and statistical information required for governmental approval.

18. The method of processing pharmacokinetic data having enhanced data management and exploration of claim 17, wherein said report creating step further comprises the steps of:
launching S-Plus; and
activating reporting features provided therein.

19. The method of processing pharmacokinetic data having enhanced data management and exploration of claim 14, further comprising the step of engendering a control stream for executing said second data set generating step.

20. The method of processing pharmacokinetic data having enhanced data management and exploration of claim 19, wherein said control stream further comprises controls for generating a plurality of data sets.
